(19) United States
(12) Patent Application Publication
(10) Pub. No.: US 2011/0288411 A1
(43) Pub. Date: Nov. 24, 2011

(54) Multi-Mode Induced Acoustic Imaging Systems And Methods

(76) Inventors: Stephen Anthony Cerwin, Mico, TX (US); David B. Chang, Tustin, CA (US); Jane F. Emerson, Irvine, CA (US)

(21) Appl. No.: 12/913,586

(22) Filed: Oct. 27, 2010

Related U.S. Application Data
(63) Continuation-in-part of application No. 12/786,232, filed on May 24, 2010.

(51) Int. Cl. A61B 18/18; A61B 8/00

(52) U.S. Cl. 600/439

(57) ABSTRACT

A multi-mode Electro-Magnetic Acoustic Imaging (EMAI) system is disclosed. The EMAI system utilizes an electromagnetic energy source to induce multiple acoustic signals in surrounding objects including a target tissue area or a transducer. The induced acoustic signals can be collected and converted to imaging data, which can be used to display a tissue area image. The collected acoustic signals can be filtered or isolated based on one or more signal proprieties including frequency. A signal's frequency can indicate a property of the target tissue including the tissue's conductivity or its density.

This application is a continuation-in-part of U.S. patent application Ser. No. 12/786,232 filed on May 24, 2010. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is acoustic imaging technologies.

### BACKGROUND

Conventional acoustic imaging systems require a transducer configured to emit acoustic signals toward a target tissue. Interactions of the emitted acoustic signals and the tissues can cause diagnostic acoustic signals representative of the target tissue to be generated. Conventional imaging devices collect the diagnostic acoustic signals and convert such signals into imaging data for display. One example includes a pre-natal ultrasound imaging device.

A lesser known technique for imaging is known as “Electro-Magnetic Acoustic Imaging” (EMAI) where electromagnetic (EM) fields bathe a target tissue rather than bathing the target tissue with an ultrasound signal or other acoustic signal. The fields can induce the target tissue to generate an acoustic signal due to conductivity gradients present in the tissue observation area. The acoustic signals originating from the tissue can also be used to generate images of the target tissue, where the images are representative of tissue conductivity rather than merely representative of tissue density. Such approaches are described in the Applicant's previous patent filings including U.S. Pat. No. 6,535,625 to Chang et al. titled “Magneto-Acoustic Imaging” filed Sep. 24, 1999, and U.S. Pat. No. 6,974,415 to Cerwin et al. titled “Electromagnetic-Acoustic Imaging” filed on May 23, 2003.

Other approaches also seek to induce acoustic signals in tissues. For example, U.S. patent application publication 2005/0107692 to Li et al. titled “Multi-Frequency Microwave-Induced Thermoacoustic Imaging of Biological Tissue”, filed Nov. 17, 2003, describes using microwave pulses swept across a range of frequencies to cause the tissue to generate theromacoustic signals. Additional work on microwave-induced acoustic signals is discussed in the paper by Wang et al. title “Microwave-induce acoustic imaging of biological tissues”, published September 1999 in Volume 70, Number 9, of Review of Scientific Instruments.

Imaging techniques have largely focused on managing a single modality (e.g., electromagnetic fields, MRI, etc.) or another (e.g., ultrasound). Still, others have attempted to combine multiple modalities into a single imaging system. U.S. patent application publication 2006/0258941 to Cable et al. titled “Multi-model internal imaging”, filed Jul. 12, 2006, discusses using light along with a second type of imaging that could include MRI, CT, or ultrasound techniques. Another examples include U.S. patent application publication 2007/0015993 to Ciocan et al. titled “Microwave imaging assisted ultrasonically”, filed Jul. 13, 2005, where microwave energy is used in concert with ultrasonic for investigating tissues. Still, others simply combine ultrasound with electromagnetic energies as discussed in U.S. patent application publication 2007/0276240 to Rosner et al. titled “System and method for imaging a target medium using acoustic and electromagnetic energies”, filed May 2, 2006. In a similar vein International patent application publication WO 2009/037710 to Harel et al. titled “MRI probe”, filed Sep. 21, 2008, discusses combining MRI with other forms of imaging techniques, including ultrasound.

Interestingly, efforts to combine different imaging techniques treat each modality distinctly where the different energies (e.g., acoustic energy, electromagnetic energy, etc.) are generated separate from each other and are typically protected from each other. Consider MRI systems; MRI systems require Radio Frequency (RF) shielding to reduce induced eddy currents in surround equipment or objects. Induced currents can wreak havoc within devices configured to emit alternative imaging energies (e.g., acoustic energy).

Conventional approaches attempt to shield acoustic generators. For example, even in the Applicant's own work described in U.S. patent application 2007/0038060 to Cerwin at al. titled “Identifying and Treating Bodily Tissues using Electromagnetically Induced, Time-Reversed, Acoustic Signals”, filed Jun. 9, 2006, the Applicant discussed a requirement for shielding to protect an ultrasonic sensor array. In a somewhat similar vein, U.S. patent application publication 2007/0167705 to Chaing et al. titled “Integrated Ultrasound Imaging System”, filed Aug. 2, 2006, discusses an ultrasound and MRI system where the ultrasound transducer or other components are shielded from electromagnetic interference.

What has yet to be appreciated is that exposing an acoustic transducer to electromagnetic energy can be desirable. As in EMAI scenarios where EM fields can induce tissues to generate induced acoustic tissue signals, the EM fields can also induce a transducer to generate induced acoustic transducer signals. The induced transducer signals can also be used for imaging purposes (e.g., diagnosis, therapy, etc.). Thus an EMAI based system can utilize both the induced tissue signals and the induced transducer signals to generate imaging data of a tissue site. By leveraged multiple modes of induced acoustic signals, one can increase the diagnostic or therapeutic efficacy of EMAI devices.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Thus, there is still a need for multi-mode induced acoustic imaging devices.

### SUMMARY OF THE INVENTION

The inventive subject matter provides apparatus, systems and methods in which one can collect multiple acoustic signals induced by electromagnetic energy and convert the acoustic signals into an image representative of a target tissue. One aspect of the inventive subject matter includes a multi-mode induced ultrasound imaging system comprising a transducer, possibly an array of transducer elements, an Electro-Magnetic (EM) Radio Frequency (RF) source, and an acoustic imaging module. The transducer can be configured to generate ultrasound signals in response to the RF energy emitted by the RF source where the induced transducer signal can be driven directly by the RF energy. The RF energy can also be directed toward a target tissue, possibly internal to a patient. The RF energy can also produce an induced tissue acoustic signal due to conductivity gradients within the tissue area. As the induced transducer signal interacts with the target tissue, a returned signal is formed, which can be collected along with the induced tissue signal. Both signals can be collected by the acoustic imaging module and converted to imaging data. A display can then present the imaging data as an image of the tissue area. In more preferred embodiments, the transducer lacks significant shielding from the RF energy to allow the RF energy to impinge on the transducer substantially unimpeded counter to known approaches. In some embodiments, the system can be configured to filter one or more of the collected acoustic signals based on various aspects of the captured acoustic data including a frequency centered on a dominate frequency of the RF energy. The RF energy can be further configured to emit RF energy having two or more dominate frequencies, or peaks. The resulting induced acoustic signals, both in the transducer and the tissue, can be analyzed based on the frequency peaks. For example, collected acoustic data could be filtered based on harmonics of the peaks, non-harmonic signals, sums of the peaks, differences of the peaks, or other filtering algorithm.

The inventive subject matter also includes a method of displaying an acoustic image. The method can include directing EM RF energy toward a target tissue as well as an acoustic transducer, possibly an ultrasound transducer array. The RF energy can induce multiple induced acoustic signals. For example, the RF energy can cause an induced transducer acoustic signal due to the interaction of the RF energy and transducer elements and cause an induced tissue acoustic signal due to the interaction of the RF energy and conductivity gradients in the tissue. The method can further include directing the induced transducer acoustic signal toward the tissue area. Yet another step can include collecting a reflection of the induced transducer signal as well as the induced tissue signal originating from the tissue area where both collected signals represent different information relating to the tissue area. The collected tissue area signals (e.g., the induced tissue signal and the returned induced transducer signal) can be converted into imaging data representing the tissue area, which can then be displayed as an image of the target area. One should appreciate that the RF energy can comprise one or more dominate frequency peaks. In some embodiments, the energy bathes both the transducer and tissue area while in other scenarios RF energy having different dominate frequencies can be split so that a first component of the energy having a first peak impinges the transducer while a second component of the energy having a second peak impinges the tissue. Providing for multiple dominate frequency peaks allows for selectively displaying image data or filtering collected acoustic tissue signals based on frequencies (e.g., the sum of the peaks, difference of the peaks, non-harmonic peaks, harmonic peaks, etc.). In more preferred embodiments, the inventive subject matter can also include averaging collected acoustic tissue signals over multiple RF energy pulses to yield image data.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an overview of an ultrasound imaging system configured to display images derived from induced acoustic signals.

FIG. 2 is a schematic overview of an imaging engine of an EMAI device.

FIG. 3 is a possible method of displaying an acoustic image.

### DETAILED DESCRIPTION

It should be noted that while the following description is drawn to a computer-based ultrasound imaging engine, various alternative configurations are also deemed suitable and may employ various computing devices including servers, interfaces, systems, databases, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to disclose apparatus. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, the Internet, LAN, WAN, VPN, or other type of networks.

One should appreciate that the disclosed techniques provide many advantageous technical effects including generating induced acoustic transducer signals that can be combined with induced acoustic tissue signals to generate images of tissues. Images derived from different modes of acoustic signals offer insight into different characteristics of a target tissue. For example, multi-mode induce acoustic imaging can provide imaging data reflective of tissue density as well as conductivity.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

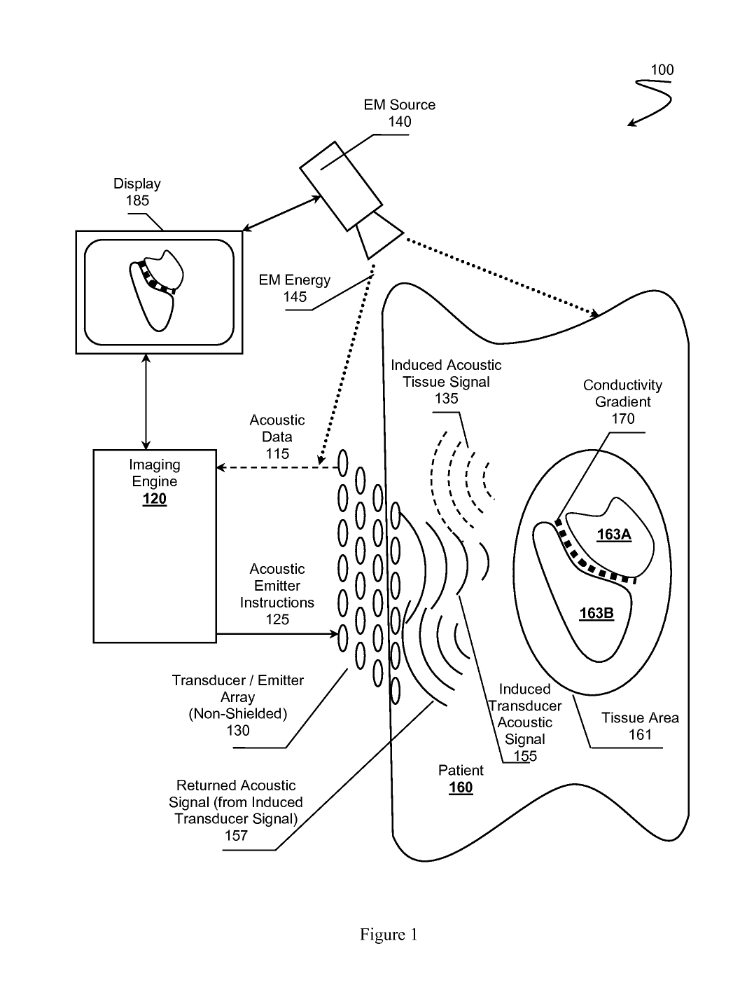

In FIG. 1, imaging system 100 comprises components to induce multiple acoustic signals that can be used to generate image data associated with target tissue area 161 within patient 160. In some embodiments, at least some of the components can be combined to form single integral devices, even a portable device. In other embodiments the components can be physically distributed where the components exchange data over a communications network, wired or wireless. The components can communicate with other components over a wired network to reduce possible EM interference. It is also contemplated the components could communicate over an optic fiber network to reduce metal components. Such an approach is considered an advantage when the components are used within conjunction of a strong magnetic field generator, possibly a coil within EM source 140.

A brief theoretical overview will aid the reader in understanding the disclosed concepts. System 100 can comprise EM source 140 that emits RF EM energy 145 toward tissue area 161 within patient 160, where EM 145 can have one or more frequency peaks (e.g., a peak at frequency f). In the example shown, tissues 163A or 163B within tissue area 161 can comprise one or more of conductivity gradient 170, typically located at a boundary between tissues. As EM energy 145 impinges on gradient 170, EM energy 145 induces gradient 170 to generate an acoustic signal 135 (e.g., ultrasound) at twice the frequency (2f) of EM energy 145. Induced acoustic tissue signal 135 can be detected via transducer 130. Furthermore, EM energy 145 can also induce transducer 130 to generate induced acoustic transducer signal 155, which can be directed toward tissue area 161. Induced acoustic transducer signal 155 comprises the same frequency (f) as EM energy 145. Transducer signal 155 interacts with tissue area 161 (e.g., tissues 163A, 163B, etc.), which causes returned acoustic signal 157 to radiate from tissue area 161 still at the same frequency (f) as signal induced transducer signal 155. Thus, induced tissue signal 135 representing a conductivity perspective of tissue area 161 can be distinguished from returned acoustic signal 157 representing a density perspective of tissue area 161. The two signals, or other induced acoustic signals can be isolated because the collected acoustic signals comprise different frequency signatures, 2f and f respectively.

The term “returned” is used euphemistically to represent a signal after an interaction between an input signal (e.g., induced acoustic signal 155) and a target (e.g., tissue area 161, tissue 163A, tissue 163B, etc.). For example, “returned induced transducer signal” indicates the induced acoustic transducer signal 155 induced at transducer 130 interacts with tissue area 161 generating returned acoustic signal 157.

Transducer 130, possibly an array of ultrasound emitters, collects one or more acoustic signals including induced acoustic tissue signal 135 and returned acoustic signal 157. Transducer 130 sends acoustic data 115 representative of the captured acoustic signals to imaging engine 120. Imaging engine 120 can convert acoustic data 115 into one or more images for display on display 185. Transducer 130 can also be configured to operate based on acoustic emitted instructions 125 provided by imaging engine 120, possibly according to conventional ultrasound techniques.

The Applicant's previous work details various aspects of EMAI systems that can be adapted for use with the disclosed techniques as discussed in co-owned U.S. Pat. No. 6,535,625; U.S. Pat. No. 6,974,415 to Cerwin et al.; and U.S. patent application publication U.S. 2007/0038060 to Cerwin et al.

Of particular note, transducer 130 lacks EM shielding that would otherwise substantially impede EM energy 145 from impinging on one or more of transducer 130. Such a configuration is desirable to allow induction of induced acoustic transducer signal 155. One should note that transducer signal 155 is generated as a property of elements within transducer 130 rather than being driven solely by electronics. For example, transducer 130 can include piezoelectric elements that are naturally responsive to EM energy 145 at various frequencies. In response, the piezoelectric elements emit acoustic signals at a commensurate frequency to the imaging EM energy 145.

EM source 140 is configured to emit RF signals toward tissue area 161. Preferred RF signals comprise a frequency in the range from 1 MHz to 500 MHz, more preferably from 1.0 MHz to 400 MHz, and yet more preferably from 1 MHz to 20 MHz. EM source 140 can also take on many different forms including a Helmholtz coil, a magnetic resonance imaging system, or other apparatus capable of generating the desirable RF signals. Furthermore, EM source 140 can be configured to emit two or more distinct signals (e.g., RF signals having distinct peaks) or a spectrum of signals, which can be used for diagnostic or therapeutic purposes.

EM source 140 can be configured to direct multiple, different RF signals toward various directions as well. For example, in some scenarios it would be advantageous to direct a first signal having a first frequency peak toward transducer 130 while directing a second signal having a different, second frequency peak toward tissue area 161. Such an approach ensures the collected acoustic signals can be easily separated from each other because induced acoustic transducer signal 155 and its returned signal 157 will be centered at the first frequency peak while the induce acoustic tissue signal 135 will be centered at twice the second frequency peak. In such an approach, the two frequency peaks can be suitably chosen so they are non-harmonic relative to each other. In view of the forgoing, the inventive subject matter is considered to include managing EM energy 145 frequency signatures or spectrums and directing EM energy 145 to desired targets based on the frequency signatures.

EM source 140 can be configured to emit pulses of EM energy 145 rather than emitting a continuous stream of energy. Such an approach is advantageous to limit a total amount of energy impinging a tissue area 161. In non-biological applications, pulsing EM energy 145 might not be necessary. For example, EM source 140 can be configured to generate a 1 μs pulse of 5 MHz every millisecond. A single pulse would comprise multiple cycles of EM energy 145 that would cause induced transducer signal 155 and tissue signal 135 to be generated. In one second, imaging engine 120 can collect 1000 acoustic data samples of induced acoustic tissue signal 135 and returned acoustic signal 157. The acoustic data samples can be signal averaged to form a crisp image of tissue area 161. Such an approach can achieve axial resolutions (i.e., in the directing of propagation) of at least 5 mm and lateral resolution (i.e., orthogonal to the direction of propagation) of at least about 2 mm.

Although preferred embodiments utilize RF frequency ranges from 1 MHz to 500 MHz, it is specifically contemplated that EMAI system 100 could utilize other ranges as well. In some scenarios microwaves can be used to induced thermoacoustic signals in target tissues where microwaves are emitted by EM source 140 in the range from 1 GHz to 300 GHz. The subject matter discussed in U.S. Patent application publication 2005/0107692 to Li et al. can be suitable adapted for use according to the disclosed techniques. Even further, one could employ induced acoustic signals generated in response to laser-based EM energy 145, possibly adapted from the techniques described by U.S. Pat. No. 5,840,023 to Oraevskey et al. titled “Optoacoustic imaging for medical diagnosis”, filed Jan. 31, 1996. One should appreciate that various induced acoustic signals can provide different information relating to the properties of tissue area 161 including density, conductivity, absorption, transmission, permittivity, permeability, or other tissue properties including physical, electrical, or biological properties depending on the input EM energy 145.

Transducer 130 can be placed external to patient 160 as illustrated. Still, other embodiments are also contemplated. In some embodiments, transducer 130 can include an internal probe that can be inserted into a body cavity or even surgically implanted. For example, transducer 130 could be position on a tip of a stint, within a probe, on a catheter, or other medical device. Although transducer 130 is illustrated as in direct contact with patient 160, in some diagnosis or therapeutic embodiments one or more intermediary layers of materials between transducer 130 and patient 160 can be employed to aid in acoustic transmission of the various acoustic signals to and from patient 160.

Transducer 130 is illustrated as an ultrasound transducer array having a plurality of transducer elements, each capable of generating a portion of induced acoustic transducer signal 155. As mentioned previously, induced transducer signal 155 is a physical phenomenon resulting from the interaction of EM energy 145 and transducer elements (e.g., piezoelectric elements). Thus induced transducer signal 155 is automatically generated as a physical phenomenon and is not solely driven by acoustic emitter instructions 125 or other external electronics. Still, control over directing induced transducer signal 155 toward target tissue area 161 or controlling signal's 155 properties (e.g., amplitude, phase, direction, etc.) would be desirable. One approach to control induced transducer signal 155 can include incorporating an acoustically adjustable layer between each transducer element and patient 160 where imaging engine 120 can send emitter instructions 125 to instruct the layer to become acoustically opaque to or at least resistant to transmission of induced transducer signal 155. For example, U.S. Pat. No. 5,285,789 to Chen et al. titled “Ultrasonic transducer apodization using acoustic blocking layer”, filed Apr. 21, 1992, describes materials that attenuate ultrasounds. Such materials can be adapted for use with the inventive subject matter by causing layers of such material to physically or mechanically block emitted induced transducer signal 155 on a transducer element-by-element basis. The subject of control of induced transducer signal 155, especially in environments lacking substantial transducer shielding, will be addressed in a follow on patent filing.

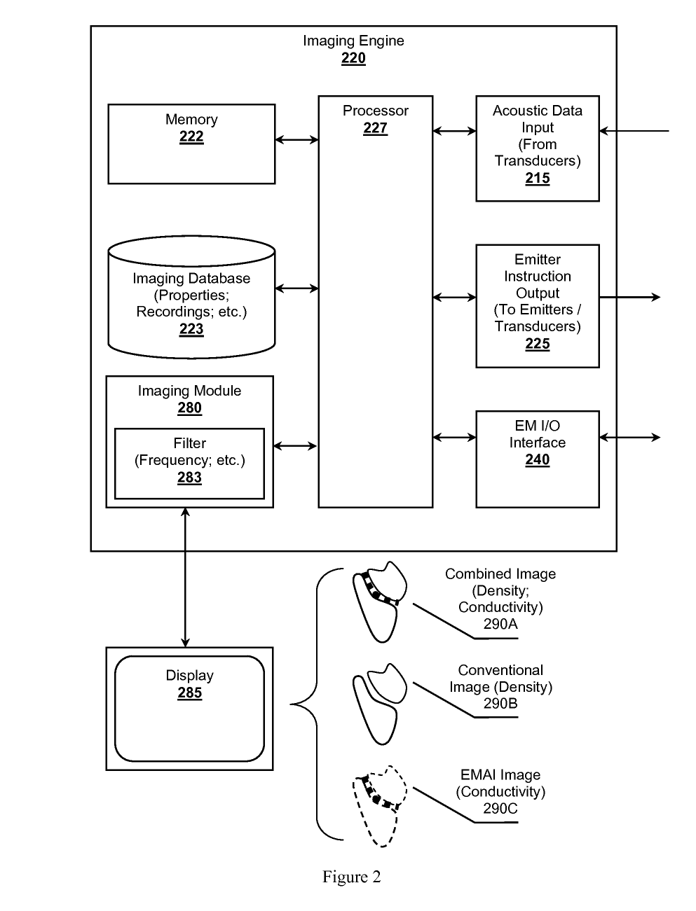

In FIG. 2 imaging engine 220 is presented in additional detail. In a preferred embodiment, engine 220 comprises a computing device having processor 227 capable of executing one or more software instructions stored in memory 222. In some embodiments, engine 220 can be a desktop computer while in other embodiments engine 220 can be a handheld device, portable device, or medical device. Regardless of the physical form of engine 220, engine 220 operates to receive acoustic data from a transducer, analyze the acoustic data, convert the acoustic data into image data, or generate instructions for the transducer to emit a treatment signal. Image data can be displayed one or more of display 285.

Imaging engine 220 can also include imaging database 223, possibly implemented within a portion of memory 222, where additional information or data can be stored. In some embodiments, database 223 stores information relating to acoustic imaging. For example database 223 could store patient data, known types of tissues and their properties, treatment parameters, therapy regimes, programmed therapies, or other type of information that can be used to analyze or display image data derived from induced acoustic signals. It is also contemplated that database 223 can be used to record sessions, image data derived from inbound acoustic data, transducer array data, management information relating to each transducer, or any other additional information.

Acoustic data input 215 represents an I/O interface to a transducer, possibly a transducer array, through which imaging engine 220 receives acoustic data collected by the transducers. Data input 215 could comprise a wired or wireless interface as desired, while a physical wired interface would be more preferable. In some embodiments, data input 215 could comprise a network connection to a remote array, possibly over the Internet. Contemplated interfaces that could be utilized for data input 215 include analog interfaces, digital interface, serial interfaces, Ethernet interfaces, or other types of interfaces for receiving data.

Collected acoustic data can be analyzed according to any desired imaging algorithm. Preferably, the acoustic data is analyzed to derive imaging data representative of a target tissue area, where the imaging data represents properties reflective of different tissue properties (e.g., density, conductivity, optical absorption, etc.) based on multiple modes of induced acoustic signals. Furthermore, information stored within imaging database 223 can also be used in conjunction with the collected acoustic data to derive an appropriate image. For example, acoustic data could be collected from a subject area having a tumor. Engine 220 uses the acoustic data to develop a three dimensional model of the tumor and surrounding areas based on a measured conductivity topology of the tumor and surrounding area. Engine 220 can consult database 223 to determine a type of tissue, preferably based on stored known acoustic, conductivity, or other characteristics of tissues. The controller can then combine the conductivity image information with density information from additional acoustic signals to display an image of the tissue area, including the tumor.

Imaging engine 220 can use information from imaging database 223 to provide additional information to a technician. For example, engine 220 can annotate regions of interest on a display, highlight specific areas, provide auditory indicators, or other information to help guide a technician operating engine 220.

Imaging engine 220 can also include one or more user interfaces (not shown) through which a technician or other user can supply input to or receive output from engine 220. Engine 220 could use imaging module 280 to provide image data to display 285. User interfaces allow a technician to guide a specific treatment via keyboard, pointer devices, or other inputs. The user input can also be used to modify acoustic signals, possibly through selecting a target area, increasing or decreasing amplitude, exposure time, or other treatment parameters. Furthermore, engine 220 can also include I/O interface 240 configured to exchange data with an EM source. Such an embodiment provides for cooperation between devices when gathering data regarding a target tissue.

Imaging module 280 is configured to collect a tissue area acoustic signal preferably representing acoustic signals induced from an EM RF source. For example, imaging module 280 can collect an induced acoustic tissue signal and a returned induced transducer signal as discussed previously, where each signal reflects different properties of the target site (e.g., conductivity, density, etc.). Module 280 can further convert collected acoustic data into imaging data using known techniques, with the expectation that the acoustic data can be isolated by frequency. For example, an EM RF signal having frequency f could cause the acoustic data to have a peak at f representing density information and have a peak at 2f representing conductivity information. Other types of induced signals can also contribute to identifying tissue properties including thermoacoustic signals, optoacoustic, or other induced acoustic signals generated externally or internally.

Imaging data can be sent from imaging module 280 to display 285 for presentation to a technician or other individual. In view of imaging data comprising information relating to different properties of a target tissue, display 285 can be configured to selectively display the tissue according to the different properties. As shown, display 285 could display combined image 290A representing multiple properties of the tissue, conductivity and density for example. Additionally, a user could select an alternative view, possibly separating the image data according to property type as indicated by images 290B and 290C. Image 290B represents a conventional ultrasound image that presents density-based information derived from returned induced transducer signals. Image 290C represents an EMAI image that presents conductivity-based information derived from induced tissue signals. Naturally other properties could also be displayed in image form including physical properties, electrical properties, biological properties, or other types of characteristics.

Selection of image data can be achieved through appropriately configuring one or more of filter 283. As mentioned previously, tissue properties can be selectively displayed by filtering based on frequency of acoustic data resulting from one or more induced acoustic signals. Although an EM source could emit an EM RF energy having a single dominate frequency peak that can be adequately used to differentiate between tissue properties, it is also considered advantageous to also direct EM RF energy having multiple, different frequency peaks toward a target tissue area or transducer. When a single frequency peak is generated, the signals include harmonics that can include undesirable noise. If non-harmonic frequency peaks are generated, then the signals can be further isolated, although harmonic noise could still be present.

When a single the EM energy comprises a single frequency peak, filter 283 can filter acoustic signals as a function of the peak. In EMAI systems as discussed above, acoustic signals resulting from an induced acoustic transducer signal can be selected based on having a frequency approximately the same of the frequency peak. Such a filter will collect data representative of a tissue's density. Acoustic signals resulting from induced acoustic tissue signals can be selected based on those signals have twice the frequency of the peak, the collect signal represents conductivity. Thus filter 283 can be configured to selectively remove acoustic data falling outside a window around the frequency peak, or around a harmonic of the frequency peak (e.g., twice the peak's frequency).

Similarly filter 283 can also be configured to remove acoustic data according to other functions as well. When the EMAI system utilizes multi-peaked input EM energy, filter 283 can selectively remove data representative of signals falling outside a window around a sum or difference of the peaks. Such an approach allows for signal separation or isolation in addition to further characterizing tissues.

To facilitate filtering or selectively accessing acoustic data, imaging module 280 can include a user interface through which a user can enter desired parameters of a filter function within filter 283. As mentioned previously, one can configure filter 283 to filter based on a sum or difference of the input frequency peaks. Other parameters can also be selected beyond frequency including phase, time of flight, amplitude, origin of signal, or other property of the collected acoustic signals.

Collected acoustic signals, filtered or unfiltered, can also be Time-Reversed Mirrored (TRM) to send a signal back toward the origin of the collected signals. The TRM signals can be amplified to create a signal of sufficient energy to internally treat a target tissue as discussed in the parent U.S. patent application having Ser. No. 12/786,232. Furthermore, the TRM signals can be finely control by filtering based on various properties of the target tissue as determined from the collected signals. For example, a healthcare provider could target tissue based on both a tissue's conductivity and the tissue's density as well as each property individually.

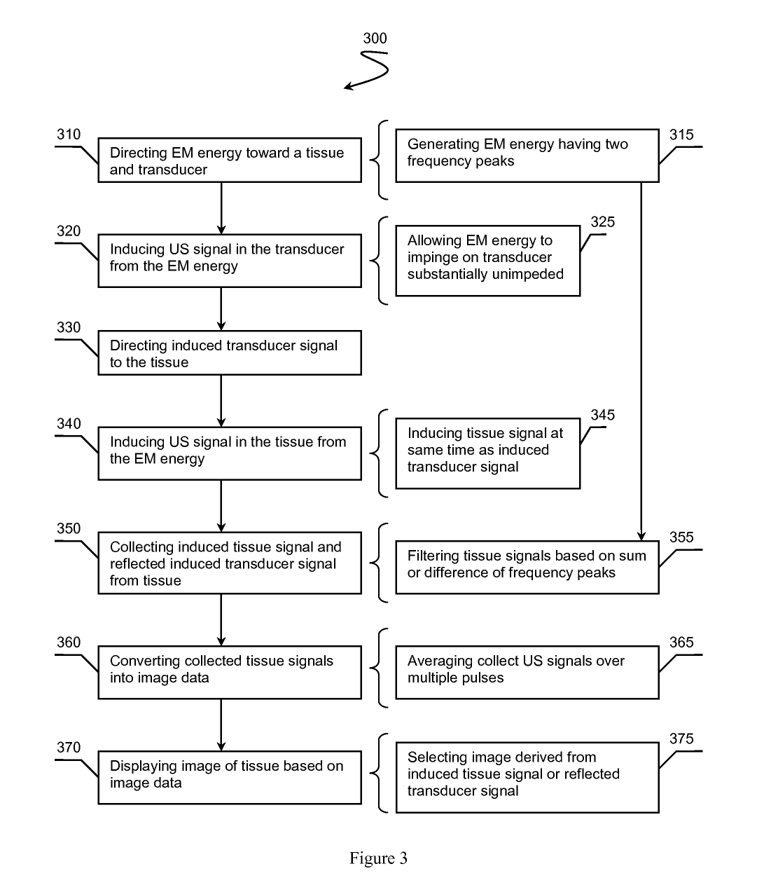

FIG. 3 provides an outline of method 300 of displaying an ultrasound image. Step 310 includes directing EM energy toward a target tissue area and a transducer. The EM energy could be generated within one, two, or more frequency peaks as indicated by step 315 where the EM energy bathes the target tissue and the transducer. In some embodiments, EM energy having a first frequency peak can be directed to the target tissue area while EM energy having a second, different frequency peak can be directed to the transducer, where the induced signals are non-harmonic.

Step 320 can include inducing an acoustic signal in the transducer. As the EM energy impinges the transducer, or transducer elements in an array, the transducer generates an ultrasound signal of the same frequency as the input EM energy. Step 325 can include allowing the EM RF energy to impinge the transducer in a substantially unimpeded fashion. It some embodiments some elements of a transducer array might be shielded while others are unshielded to allow for control over how specific elements in the array emit their induced acoustic transducer signals.

Step 330 includes directing the induced acoustic transducer signal toward a target tissue. The induced transducer signal can interact with a target tissue to form a returned induced transducer signal, which can then be collected an analyzed.

Step 340 includes inducing an acoustic signal in a target tissue where the EM energy causes the tissue to generate an ultrasound signal of approximately twice the frequency of the input EM energy. The induced acoustic tissue signal is generated due to the interaction of the EM energy with a conductivity gradient of the tissue. Step 345 can further include inducing the induced tissue signal at the same time as the induced transducer signal. Other embodiments can pulse each induction target (e.g., the tissue, the transducer, etc.) independently if desired to further isolate the two signals in time.

Step 350 can include collecting the induced tissue signal and the returned induced transducer signal. The various induced signals, or returned versions of the induced signals, can also be filtered if desired based on their signal characteristics including frequency, phase, amplitude, time of flight, origin or other properties. In embodiments where the input EM energy includes more than one frequency peak as suggested by step 315, method 300 can further include step 355. Step 355 includes filtering acoustic tissue signals (e.g., induce acoustic tissue signals, returned induced acoustic transducer signals, etc.) based on sums or differences of frequency peaks.

Step 360 can include converting collected acoustic tissue signals into image data. In some embodiments, an EM source generates short pulses of RF EM energy. For example, the EM source could generate a 1 us pulse of a 5 MHz RF signal. The pulses can be generated according to a desired periodicity, every 1 ms for example. The system can collect acoustic data resulting from induced signals generated every millisecond. The system can further average the collected signals over multiple EM pulses to generate a stronger image of a target tissue area as indicated by Step 365.

Step 370 can include displaying an image of the target tissue area based on the image data resulting from analysis of the induced collected acoustic signals. In some embodiments, an EMAI imaging system provides a user interface through which a healthcare provider is able to selectively display tissue area information. For example, Step 375 can include selecting an image derived from returned induced transducer signals or derived from induced tissue signals, each selected signal reflective of different tissue properties.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A multi-induced ultrasound imaging system, the system comprising:
a transducer configured to generate an induced ultrasound transducer signal as induced by RF energy, and configured to direct the induced ultrasound transducer signal to the tissue area;
an RF energy source configured to generate and direct RF energy to a tissue area and to the transducer array;
an acoustic imaging engine configured to:
(a) collect a returned induced ultrasound transducer signal reflective of an interaction between the induced ultrasound transducer signal and the tissue area, and an induced ultrasound tissue signal induced by the RF energy and originating from the tissue area, and
(b) convert the returned induced transducer signal and the induced tissue signal into an ultrasound imaging data representative of the tissue area; and
a display configured to obtain the imaging data and to display an image of the tissue area based on the imaging data.

2. The system of claim 1, wherein the display is configured to selectively display the image as represented by only one of the following: the returned induced transducer signal and the induced tissue signal.

3. The system of claim 1, wherein the tissue area is internal to a patient.

4. The system of claim 1, wherein the transducer array lacks substantial shielding to allow the RF energy to impinge the transducer array.

5. The system of claim 1, wherein the acoustic imaging engine is configured to filter the collected returned induced transducer signal and induced tissue signal according to a filter function based on at least one frequency of the RF electromagnetic energy.

6. The system of claim 5, wherein the filter function removes ultrasound signals outside a window around at least one harmonic of the at least one frequency.

7. The system of claim 6, wherein the at least one harmonic is twice the at least one frequency.

8. The system of claim 5, wherein the filter function removes ultrasound signals outside a window around a sum or a difference of two RF input frequencies.

9. The system of claim 5, wherein the acoustic imaging engine comprises a filter interface through which a user can enter parameters of the filter function.

10. The system of claim 1, wherein the transducer array comprises piezoelectric elements that generate the induced ultrasound transducer signal in response to the RF energy interacting with the elements.

11. The system of claim 1, wherein the returned induced transducer signal comprises a first frequency peak and the induced tissue signal comprises a second, different frequency peak.

12. The system of claim 11, wherein the second frequency peak is approximately twice the first frequency peak.

13. A method of displaying an ultrasound image, the method comprising:
directing RF electromagnetic energy toward a tissue area and an ultrasound transducer array;
inducing an induced ultrasound transducer signal in the transducer array based on the RF energy;
directing the induced ultrasound transducer signal to the tissue area;
inducing an induced ultrasound tissue signal within the tissue area based on the RF energy;
collecting a returned induced ultrasound transducer signal reflective of an interaction between the induced ultrasound transducer signal and the tissue area, and the induced ultrasound tissue signal originating from the tissue area;
converting the return induced transducer signal and the induced tissue signal into a tissue area image data; and
displaying an image of the tissue area based on the tissue area image data.

14. The method of claim 13, wherein the step of directing the RF energy includes generating electromagnetic energy having at least two frequency peaks.

15. The method of claim 14, wherein the two frequency peaks are non-harmonic.

16. The method of claim 14, further comprising filtering the returned induced transducer signal and induced tissue signal based on at least one of a sum and a difference of the at least two frequency peaks.

17. The method of claim 13, wherein the step of displaying the image includes selecting between image information derived from the returned induced transducer signal and image information derived from the induced tissue signal.

18. The method of claim 13, further comprising allowing the RF electromagnetic energy to impinge on the transducer array without substantial interference.

19. The method of claim 13, wherein the steps of inducing the induced ultrasound transducer signal and the induced ultrasound tissue signals occur substantially at the same time.

20. The method of claim 13, wherein the step of converting the return induced transducer signal and induced tissue signal includes signal-averaging the collected ultrasound signals over multiple RF energy pulses.
